(19) United States
(12) Patent Application Publication
(10) Pub. No.: US 2011/0312080 A1
(43) Pub. Date: Dec. 22, 2011

(54) POROUS FILMS BY A TEMPLATING CO-ASSEMBLY PROCESS

(75) Inventors: Benjamin Hatton, Cambridge, MA (US); Joanna Aizenberg, Boston, MA (US)

(73) Assignee: PRESIDENT AND FELLOWS OF HARVARD COLLEGE, Cambridge, MA (US)

(21) Appl. No.: 13/058,611

(22) PCT Filed: Aug. 26, 2009
(86) PCT No.: PCT/US09/55044

§ 371 Date: Sep. 9, 2011

Related U.S. Application Data
(60) Provisional application No. 61/091,941, filed on Aug. 26, 2008.

(51) Int. Cl. B32B 3/26; C23C 16/44; B05D 3/12; B01J 32/00; B05D 3/00; B32B 5/16; B32B 5/22; H01M 2/14; C12M 3/00; B05D 1/18

(52) U.S. Cl. 435/289.1; 427/248.1; 427/240; 427/430.1; 427/271; 428/323; 428/221; 428/338; 428/317.9; 429/516; 502/439

(57) ABSTRACT

A method of making a composite includes providing a particle suspension comprising colloidal particles (430) and a soluble matrix precursor (440); and co-depositing the particles and the matrix precursor on a surface in a process that provides a composite of an ordered colloidal crystal comprised of colloidal particles (430) with interstitial matrix (440). Optionally the templated colloidal particles can be removed to provide a defect-free inverse opal structure.

### RELATED APPLICATIONS

This application claims the benefit of priority under 35 U.S.C. §119(e) to copending U.S. Provisional Application No. 61/091,941, filed Aug. 26, 2008, and entitled “NANOPOROUS FILMS BY A COLLOIDAL CO-ASSEMBLY PROCESS, which is hereby incorporated in its entirety by reference.

### STATEMENT REGARDING FEDERALLY SPONSORED RESEARCH OR DEVELOPMENT

This invention was made with U.S. Government support under N00014-07-1-0690 awarded by the Office of Naval Research. The U.S. Government has certain rights in the invention.

### COPYRIGHT NOTICE

This patent disclosure may contain material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### INCORPORATION BY REFERENCE

All patents, patent applications and publications cited herein are hereby incorporated by reference in their entirety in order to more fully describe the state of the art as known to those skilled therein as of the date of the invention described herein.

### BACKGROUND

Colloidal crystals are solid aggregates of colloidal particles (i.e. spheres having diameter<1000 nm) packed in ordered, crystalline structures which are typically close-packed. An example of a multi-layered film of 300 nm diameter polymer (PMMA) spheres, deposited in a close-packed array, is shown in FIG. 1A. There are many examples of how these highly-periodic, ordered structures can be fabricated by self-assembly, and used as templates to make porous “inverse” structures, by infiltrating with a secondary matrix material within the interstitial space (FIG. 1B). Known as ‘inverse opals’, these highly porous, ordered structures have been synthesized for a wide range of materials, including ceramics, polymers and metals.

Colloidal crystal films prepared by conventional methods (i.e., evaporation or sedimentation) typically have ordered crystalline domains only over relatively short lengths, typically ˜10 to 100 μm, thereby limiting the potential applications of the films. The crystal structure of these films is normally face-centered cubic (FCC), with the (111) plane oriented parallel to the surface. Typically defects limit the size and uniformity of these individual crystalline domains, or grains. The most common defects are cracks and grain boundaries that exist between the ordered domains, which are oriented in different directions within the plane of the thin film. Typically, for polymer spheres of size 300 nm, conventional evaporative (EISA) methods produce ordered domains of around 10 μm in size.

Oxides such as SiO₂, TiO₂ and Al₂O₃ can be synthesized relatively easily from sol-gel chemical precursors, and have useful properties for a wide range of applications. These structures have high porosity (>75%), with interconnected pores in the range of 100 nm to 2 μm, which gives them very high available surface area. Therefore, metal oxide inverse opal materials are potentially useful for applications such as catalysis (TiO₂, ZnO, etc), scaffold structures for tissue engineering (TiO₂, Al₂O₃, hydroxyapatite), gas or biological sensors (SnO, etc), drug delivery, among many others. Another well-known scientific and technological interest for these materials is for their photonic properties, as so-called ‘photonic band gap’ materials, due to the interference of light at a given wavelength with the ordered porous structure having a similar periodic length scale

### SUMMARY

A method has been developed to deposit porous films with pore size ranging from 10¹ nm to 10³ μm, using a one-step process of co-assembly of a template of polymer colloid or bead particles with a soluble matrix precursor, e.g., a polymerizable (sol-gel) matrix. In some applications, the polymer template may be removed to form a porous structure, but for others the polymer template may remain to form a composite material. The films have highly uniform thickness, without cracks. In some embodiments, there is no formation of an overlayer cover such that the porous volume is accessible from the top surface. If monodispersed templating particles are used, the films can have pores in a highly-ordered, close-packed arrangement. In this case, the nanoporous films demonstrate large single crystalline domains on the order of millimeters and even centimeters. The crystalline order takes place over dimensions that are orders of magnitude (10,000× or more) greater than using conventionally prepared colloidal crystals.

A method to produce 3D porous films, crack-free and without an overlayer, with and without long range order, is provided. In one aspect, a method of making a composite includes providing a particle suspension comprising templating particles and a soluble matrix precursor; depositing the particles and the matrix precursor on a surface in a process that provides a composite layer of a particle assembly comprised of templating particles with an interstitial matrix.

In any of the embodiments herein, the templating particles include organic polymers, silicates or metal oxides.

In any of the embodiments herein, the templating particles have a diameter in a range of about from 50 nm to 1000 nm, or the templating particles have a diameter of up to about 2 μm, or the templating particles have a diameter of up to about 300 μm or up to about 500 μm.

In any of the embodiments herein, the soluble matrix precursor content ranges from about 0.005 wt % to about 1.0 wt %, or wherein the templating particle content ranges from about 0.10 vol % to about 3.0 vol %.

In any of the embodiments herein, the composite assembly is a periodic, close-packed structure with long-range order, or the composite assembly has no long-range order.

In any of the embodiments herein, the method of depositing comprises evaporative induced self-assembly, and optionally the method of depositing is selected from the group consisting of sedimentation, evaporative techniques, spin coating, flow controlled deposition, shear flow reactions, or filtration.

In any of the embodiments herein, the soluble matrix precursor is selected from the group consisting of metal oxide precursors, (metal salt, metal alkoxide, silicate), calcium phosphate precursors, soluble organic polymers (polyacrylic acid, polymethylmethacrylate, cellulose, polydimethylsiloxane, polypyrrole, agarose), proteins, and polymer precursors. The matrix precursor can be soluble in aqueous or non-aqueous solvents, depending on what is used for the template suspension.

In any of the embodiments herein, the templating particles are monodisperse in size, or the templating particles contain particles of different sizes and, for example, can be a bimodal particle size distribution. In any of the embodiments herein, the templating particles include smaller nanoparticles that are smaller than the larger templating particles, and where optionally, the nanoparticles are on the range of one to two orders of magnitude smaller than the templating particles, or the nanoparticles are less than about 10 nm in diameter, or the nanoparticles are less than about 5 nm in diameter.

In any of the embodiments herein, the method further includes removing the templating particles to provide an inverse porous structure, for example, by heating to remove the templating particles, or by dissolving the templating particles, or by etching the templating particles.

In any of the embodiments herein, the concentration of templating particles and soluble matrix precurose in the particle suspension is selected to provide a substantially crack-free composite assembly that is substantially free of an overlayer of interstitial matrix material.

In another aspect, a composite is provided having a colloidal crystalline structure including periodic, close packed templating particles and an interstitial matrix, wherein the crystalline structure comprises ordered domains greater than 100 μm.

In any of the embodiments herein, the crystalline structure of the composite comprises ordered domains greater than 500 μm, ordered domains in the range of about 100 μm to about 2 cm.

In any of the embodiments herein, the colloidal crystalline structure of the composite comprises an organic polymer, or the colloidal crystalline structure comprises a metal oxide.

In any of the embodiments herein, the colloidal crystalline structure of the composite comprises templating particles having a diameter in a range of about from 50 nm to 1000 nm, or a diameter of up to about 2 μm, or a diameter of up to about 10 μm.

In any of the embodiments herein, the colloidal crystalline structure of the composite has no overlayer coating, such that the pores are open on the top surface, or the colloidal crystalline structure includes an overlayer of interstitial matrix material.

In any of the embodiments herein, the interstitial matrix is selected from the group consisting of metal oxides, organic polymers, calcium phosphates and block copolymers.

In any of the embodiments herein, the matrix of the composite comprises nanoparticles that are smaller than the particles comprising the colloidal crystalline structure, and optionally, the nanoparticles are on the range of one to two orders of magnitude smaller than the templating particles, e.g., the nanoparticles are less than about 10 nm in diameter or the nanoparticles are less than about 5 nm in diameter.

In any of the embodiments herein, the templating particles of the composite contain particles of different sizes and, for example, can be a bimodal particle size distribution. In any of the embodiments herein, the templating particles include smaller nanoparticles that are smaller than the larger templating particles, and where optionally, the nanoparticles are on the range of one to two orders of magnitude smaller than the templating particles, or the nanoparticles are less than about 10 nm in diameter, or the nanoparticles are less than about 5 nm in diameter.

In another aspect, a inverse opal layer having a porous layer is provided including an interstitial matrix defining pores, wherein the pore structure comprises ordered domains greater than 100 μm.

In one or more embodiments, the pore structure of the inverse opal layer comprises ordered domains greater than 500 μm, or about 100 μm to about 2 cm or up to about 10 cm.

In one or more embodiments, the pores of the inverse opal have a diameter in a range of about from 50 nm to 1000 nm, or pores have a diameter of up to about 2 μm, or the pores have a diameter of up to about 10 μm.

In one or more embodiments, the matrix of the inverse opal layer is selected from the group consisting of metal oxides, organic polymers and block copolymers.

In one or more embodiments, the matrix of the inverse opal layer comprises nanoparticles that are less than about 10 nm in diameter, or less than about 5 nm in diameter.

In any of the embodiments herein, porous structure of the inverse opal layer has no overlayer coating, such that the pores are open on the top surface, or the porous structure of the inverse opal layer includes an overlayer of interstitial matrix material.

In any of the embodiments herein, the porous structure of the inverse opal layer has a hierarchy of pore sizes, with large macropores in the range 1 μm to around 2 mm.

In another aspect, a sensor or scaffold for tissue engineering or fuel cell membrane or catalyst support is provided having an interstitial matrix defining a distribution of pores.

In another aspect, a photonic device is provided having a pore structure comprising ordered domains greater than 100 μm.

A technological aspect of the method, and material, is the formation of uniform, crack-free, defect-free, nanoporous layers with no overlayer over large (cm and more) area. One application will provide an inexpensive way to make porous scaffold structures for catalysis, fuel cells or sensors, with some amount of size distribution of pores that does not have long-range order. Another application is the formation of highly-ordered structures for certain applications, such as optical/photonics.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1A is a scanning electron microscope (SEM) micrograph of a colloidal crystal composed of ˜300 nm diameter polymethylmethacrylate (PMMA) particles in a conventional close-packed array.

FIG. 1B is a SEM photomicrograph of a porous “inverse opal” structure obtained by molding a material within the interstitial space of a colloidal crystal as illustrated in FIG. 1A.

FIG. 2 is a schematic illustration of the three-step process conventionally used to make inverse colloidal crystal structures.

FIG. 3A is a micrograph of a prior art PMMA colloidal crystal in low and high magnification; FIG. 3B is a prior art micrograph of a SiO₂ inverse opal films illustrating the problems of overlayer formation and cracking; and FIG. 3C is a photomicrograph of a defect-free and crack-free SiO₂/PMMA nanocomposite according to one or more embodiments of the present invention.

FIG. 4 is a schematic illustration of a two-step process according to one or more embodiments used to make inverse colloidal crystal structures.

FIG. 5 is a schematic illustration of the evaporation-induced self-assembly method used according to one or more embodiments to form an ordered nanocomposite of ordered templating particles in a metal oxide matrix.

FIG. 6A is a SEM photomicrograph of a prior art PMMA colloidal crystal in top and side views, with no added silicate matrix; and FIGS. 6B-6E show examples of SiO₂ inverse opal films (after template removal by calcination at 500° C.), in top and side views, with increasing amounts of added silicate, (i.e.; increasing SiO₂/PMMA template ratio (Scale bars=2 μm).

FIGS. 7A-7E shows examples of SiO₂ inverse opal films produced by the co-assembly method according to one or more embodiments in which FIG. 7A is a low magnification, optical photograph of a glass slide substrate coated in the porous film; FIG. 7B shows optical absorption spectra, which indicate a peak corresponding to the Bragg diffraction condition; and FIGS. 7C-E show scanning electron microscopy (SEM) images of porous SiO₂ inverse opal films, indicating the very high degree of order, without localized cracking, and without the formation of an overlayer.

FIGS. 8A-8B show examples of a SiO₂ inverse opal film deposited within the patterned channels of a Si wafer from a top view in low (8A) and high magnification (inset) and (8B) cross-section view.

FIG. 8C-8D are photomicrographs of and a SiO₂/PMMA composite film deposited around a 1 mm diameter SiO₂ glass capillary tube in low (8C) and high magnification (8D) (capillary tube is shown in inset).

FIG. 9A is a photomicrograph of SiO₂ inverse opal films deposited at different templating particle concentrations onto a surface, to control the film thickness (values represent mL of 0.125 vol % PMMA/TEOS suspension added per 20 mL H₂O) and FIG. 9B is a plot of thickness vs. solids loading for the films of FIG. 9A.

FIG. 10A shows an example of a TiO₂ inverse opal layer prepared from a TiO₂ precursor (TiBALDH); and FIG. 10B shows an organosilica inverse opal layer prepared from a silsesquioxane ((EtO)₃Si—C₂H₄—Si(OEt)₃) sol-gel precursor (high magnification is shown in inset).

FIGS. 11A-C are is a schematic illustration of a co-assembly process involving a soluble matrix (i.e.; Si(OH)₄) and template spheres of two different sizes in which smaller templating spheres (radius r₂) pack around a larger spheres (radius r₁); FIG. 11A shows the matrix (Si(OH)₄) and template spheres in suspension;

FIG. 11B shows the co-assembled composite structure as an individual sphere shell structure, before and after template removal, showing a porous SiO₂ shell with pores of sizes r₁ and r₂; FIG. 11C shows a co-assembled inverse opal structure of many larger spheres (radius r₁) on a surface, consisting of walls having smaller pores with radius r₂.

FIGS. 12A-12F are photomicrographs of 300 μm diameter porous SiO₂ shells according to the process of FIG. 11, consisting of walls having 300 nm pores.

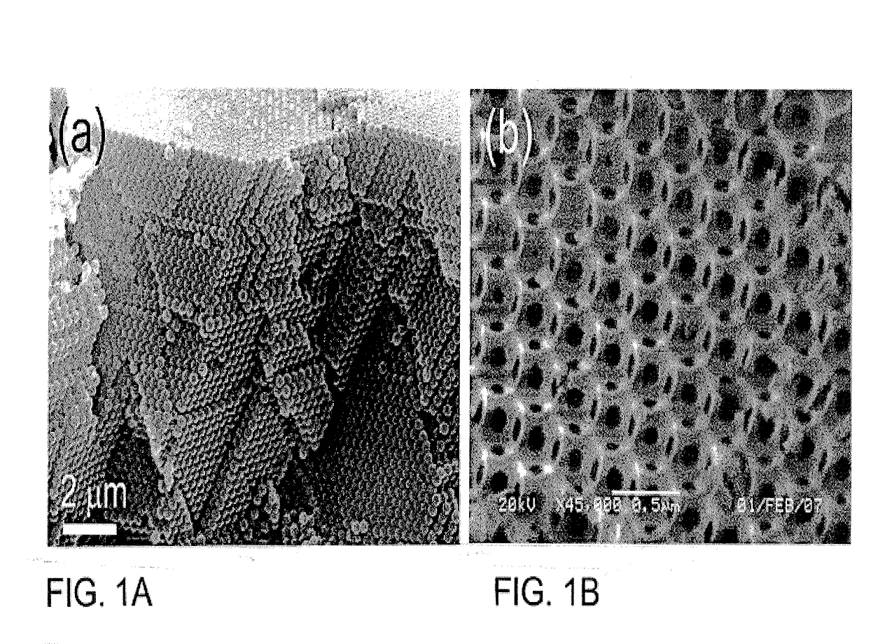

### DETAILED DESCRIPTION

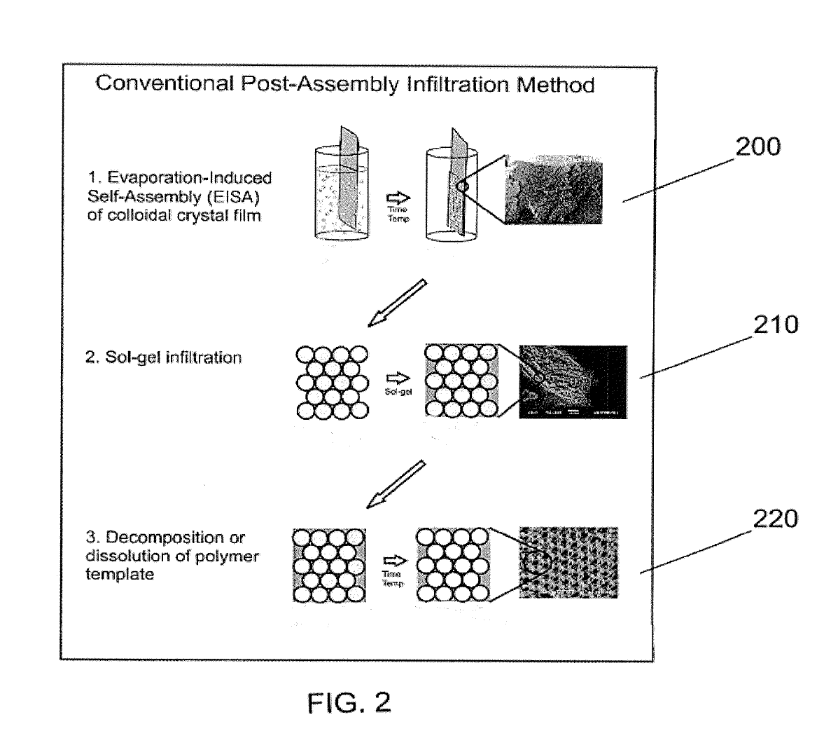

The conventional methods to make inverse colloidal crystal structures according to one or more embodiments include the following steps: (1) preparing a colloidal crystal from spherical colloidal particles, to act as a sacrificial template (step 200); and then (2) infiltrating a solution of matrix material (such as a sol-gel metal oxide precursor) into the colloidal crystal (step 210); then (3) burning away (or otherwise removing) the colloidal template, to leave an inverse porous metal oxide structure (step 220). Therefore, this is a 3-step process, illustrated schematically in FIG. 2, that involves infiltration of the metal oxide precursor after the template assembly (post-assembly infiltration). The colloidal crystal shown in FIG. 2 is formed as a thin film deposited using an evaporative induced self-assembly [EISA] method, discussed in greater detail below.

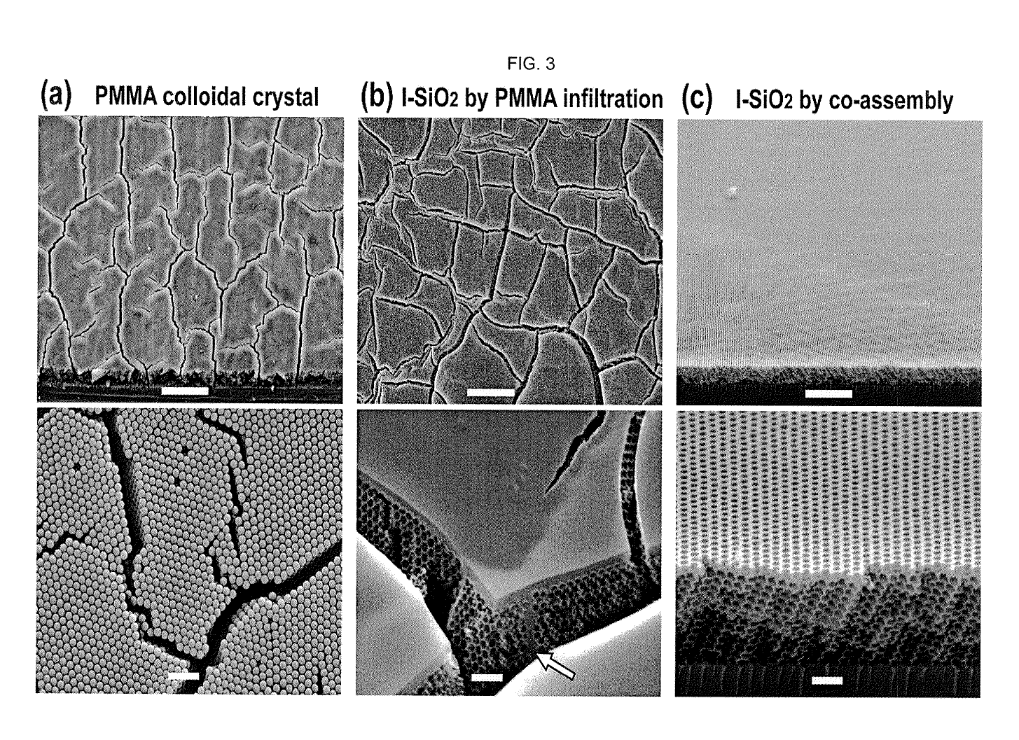

There are several problems with this conventional, post-assembly, infiltration method to make inverse opal films uniform and defect-free. Firstly, colloidal crystal films themselves are difficult to make without cracks and without many small crystallite domains of random orientation. Secondly, there are many problems associated with the secondary (infiltration) step. Particularly for the synthesis of film structures, it is difficult to uniformly infiltrate a liquid precursor over a large length scale (0.10 mm to 10 mm or beyond) of the colloidal crystal film. As a result, non-uniformity can lead to both under- or over-infiltration, leading to either structural collapse or the formation of an overlayer, respectively. Also, cracking is a major problem, due to the capillary forces associated with the infiltration of a liquid into the fragile porous colloidal crystal structure. An example of a PMMA colloidal crystal illustrating cracks is shown in FIG. 3A and a SiO₂ inverse opal film showing problems of overlayer formation, cracking and SiO₂ intrusion into the cracks is shown in FIG. 3B. SiO₂ has infiltrated cracks formed in the original colloidal crystal template, as is indicated by the arrow.

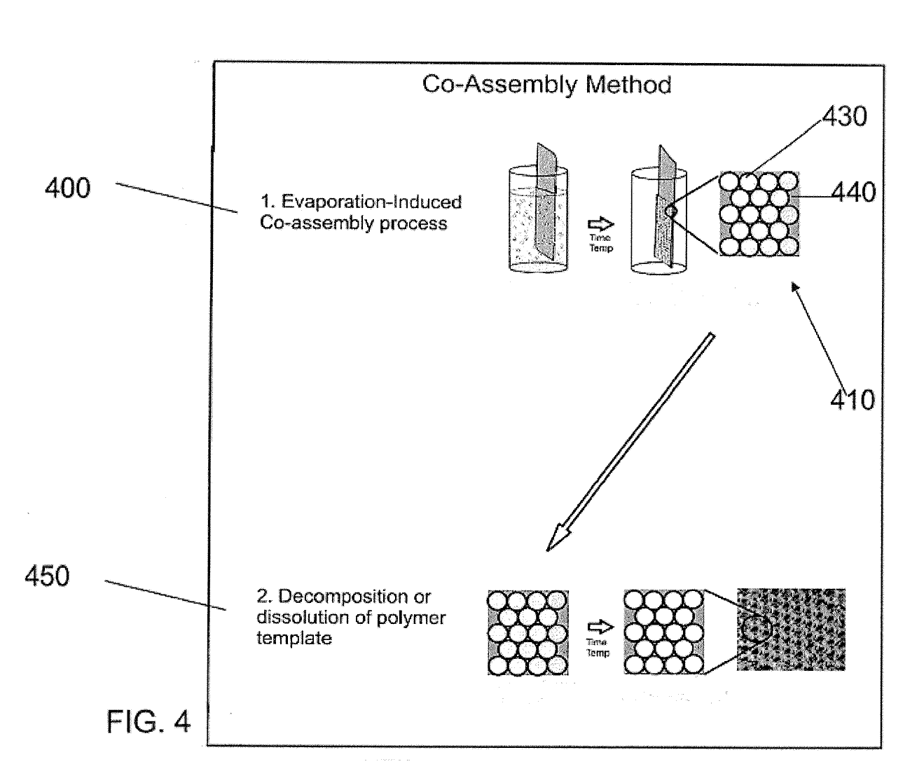

Methods are described herein to provide colloidal crystal composites and inverse opal porous structures having large crack-free domains. In one or more embodiments, colloidal crystal nanocomposites are prepared as illustrated schematically in FIG. 4. The process includes one-step co-assembly of the templating particles with a soluble matrix precursor in step 400. As a result, a composite (for example, a microcomposite or nanocomposite) film 410 is first deposited, which includes the polymer templating particles 430 in a matrix 440. The formation of the composite structure using the conventional methods requires two steps as described above. Then, the template is removed in a subsequent step 450 (to leave behind a porous matrix film made up of the matrix material). Template removal is optional and may be accomplished using a variety of methods such as thermal decomposition (burning at 300-500° C.), solvent dissolution, or oxygen plasma etching. Upon removal of the template particles, a porous structure is obtained.

In one or more embodiments, the soluble matrix material, to be co-assembled with the template particles, is a precursor to a solid material such as metal oxides or polymer, and can be a sol-gel precursor, polymer solution, or even templating particles much smaller than the template particles (i.e.; 1 or 2 orders of magnitude smaller in size). The soluble matrix precursor typically includes a polymer or a polymerizable precursor that is soluble in a carrier liquid. Very small particles, e.g., particles having a particle dimension of less than about 10 nm, can be sufficiently solvated in the carrier liquid such that they can be considered ‘soluble’ for the purposes of this process. The carrier can be aqueous or non-aqueous liquids. The carrier liquid can be a mixture or water and water-soluble organic solvents, e.g., water and a small organic alcohol. The carrier can be selected to provide balance of solubility, wetting and evaporative properties. For example, the carrier liquid could solubilize the matrix precursor, wet the surface of the depositing substrate and evaporate at a rate that allows assembly of the templating particles on the substrate.

The co-assembly of templating particles and soluble matrix precursor to form the composite 440 can be accomplished, for example, by sedimentation, spin coating, evaporative techniques, shear flow reactors, or filtration. In one or more embodiments, an evaporative technique is used. In one or more embodiments, a composite of templating particles in a metal oxide matrix is obtained using evaporative self-assembly, a technique established about 10 years ago for the deposition of colloidal crystal thin films from a particle suspension of size-monodispersed particles (i.e.; spheres). If the particle suspension contains monodispersed particles (i.e.; <5% size variation), an ordered colloidal crystal film will be formed. Otherwise, a colloidal crystal film without long-range order will be formed.

In one or more embodiments, a substrate is introduced into a dilute particle suspension, e.g., an aqueous suspension of polymer latex particles and hydrolyzed soluble sol-gel precursor, and allowed to evaporate slowly over a period of time, e.g., 1-3 days. As the solvent evaporates, the solid content, consisting of the template particles and the sol-gel material, remains behind and is deposited on the substrate as a continuous, composite thin film. Highly-ordered colloidal crystal composite films can be deposited using spheres of silica or polymer (latex) in the size range of about 10 nm to about 100 μm, and for example about 100 to 1000 nm. Following deposition, the polymer/oxide composite optionally is heated to thermally decompose the polymer template and leave behind the porous oxide film.

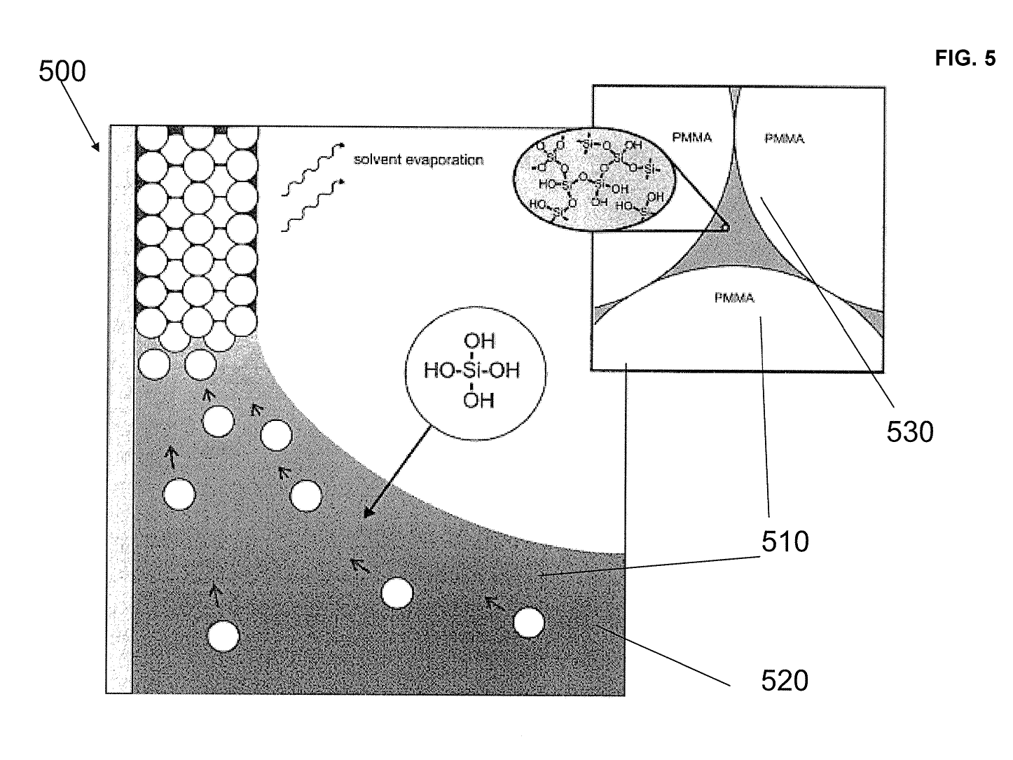

FIG. 5 shows an exemplary system 500 for the ‘co-assembly EISA’ method according to one or more embodiments. The particle suspension includes polymer template particles 510 and a soluble sol-gel precursor 520, such as the exemplary silicate matrix precursor (Si(OH)₄) shown. The sol-gel precursor can be a metal alkoxide or Si alkoxide, which is soluble in the suspension liquid and reasonably stable in solution (such as, for example, Si(OC₂H₅), tetraethylorthosilicate, TEOS). The sol-gel precursor can be partially or fully hydrolyzed (i.e.; to Si(OH)₄) in the particle suspension, or it can be an unhydrolyzed precursor. The sol-gel precursor slowly is converted into an oxide (i.e.; silica, SiO₂) during or after self-assembly of the colloidal crystal by the process of network polymerization. As a result, there is a continuous, distributed network 530 of oxide material (SiO₂) that is produced around and between the individual polymeric template (e.g., PMMA) spheres.

The substrate is withdrawn slowly from the particle suspension, or held stationary vertically as the solvent is allowed to evaporate, to provide adequate time for the template particles to self-assemble at the solid/liquid/gas interface. In addition, this time period allows the sol-gel precursor to gel, precipitate and/or polymerize as a solid matrix around and within the template particles. The solidification of the matrix may be completed during or after the template particle self-assembly process. Additional template particles or matrix precursor material, or both, can be added to the particle suspension to supplement any materials depleted during the co-assembly process. A non-aqueous solvent, such as EtOH, can be used instead of, or in addition to, an aqueous solvent to extend this method to co-deposit a wide range of material precursors that are not water-soluble.

A range of sol-gel precursors may suitably be used according to one or more embodiments to provide a metal oxide network upon hydrolysis and polymerization, or other further chemical reaction. By way of example, sol-gel precursors to SiO₂, TiO₂, Al₂O₅, ZrO₂ and GeO₂ are known and may be used as precursors according to one or more embodiments. The sol-gel precursor may be an inorganic precursor, e.g., a silicate, or it can be an organosilicate, such as tetraethyl orthosilicate (TEOS). TEOS converts readily into silicon dioxide (SiO₂) via a series of hydrolysis and condensation polymerization reactions that convert the TEOS molecule monomers into a mineral-like solid via the formation of Si—O—Si linkages. Rates of this conversion are sensitive to the presence of acids and bases, both of which serve as catalysts. Alkoxide precursors may contain reactive organic groups other than ethoxy groups. Furthermore, sol-gel precursors containing bridging organic groups (i.e.; organosilane) may be used to impart desirable properties into the final product. By way of example, the organic group can be selected for its suitability for attachment of a chemically, or biologically, functional organic group, such as an amine or carboxylic acid group, or an antibody or DNA strand, or growth factors, or other bio-inductive motifs.

In one or more embodiments, the soluble matrix precursor can be one or more of metal salts, metal oxide precursors, (metal salt, metal alkoxide, silicate), calcium phosphate precursors, soluble organic polymers (polyacrylic acid, polymethylmethacrylate, cellulose, polydimethylsiloxane, polypyrrole, agarose), proteins, alkoxysilanes, polysaccharides and polymer precursors. Suitable materials include tetraethoxysilane (TEOS), Ti butoxide, Ti isoproxide, TiO2 nanoparticles, TiBALDH (dihydroxybis-(ammonium lactato)titanium (IV)), organo silsesquioxanes, polymethylmethacrylate, polylactic acid, polyacrylic acid, epoxy polymers, agar, agarose, polydimethylsiloxane, polystyrene, polypyrrole, cellulose, collagen, hydroxyapatite, and calcium phosphates. Phenolic resin is another class of suitable matrix materials. It can be used as a matrix as it is, as an inverse opal structure to be an oil sensor. In other embodiments, it can be used as a precursor towards making a carbon structure, which is a useful catalytic material. Biopolymers also can be used as matrix precursors, e.g. agar, collagen or polysaccharides. The polymer solution occupies the interstitial spaces of the assembled colloid particles and forms a solid polymer upon solvent evaporation. In other embodiments, the soluble matrix precursor can be a polymer precursor that forms a solid matrix upon polymerization or curing. Any conventional polymers, polymerization and curing materials and methods can be used. The matrix precursor can be soluble in aqueous or non-aqueous solvents, depending on what is used for the template suspension. The soluble matrix precursor can be any soluble polymer, e.g., polystyrene, in a suitable solvent, e.g., acetone.

In one or more embodiments, the soluble precursor can be a nanoparticle that is significantly, e.g., 1-2 orders of magnitude, smaller than the templating particles. In one or more embodiments, the nanoparticle is less than 10 nm, or less than 5 nm, or in the range of about 2-5 nm. Particles of this dimension can be considered solvated by the carrier liquid. The solvent can be water or a suitable non-aqueous solvent.

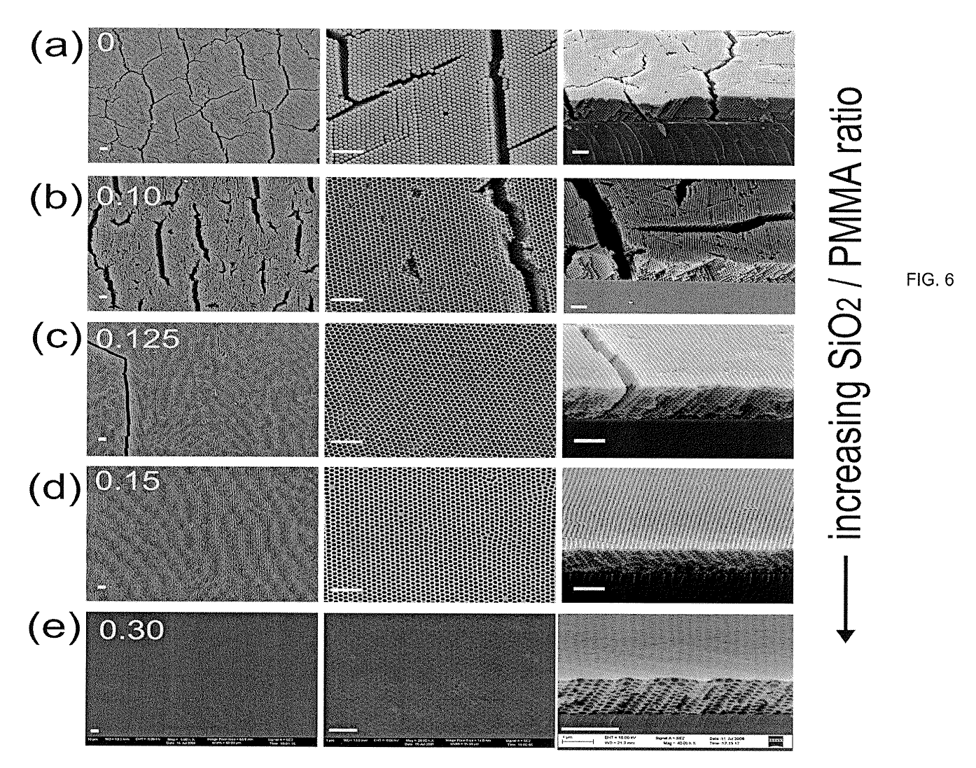

The soluble matrix precursor concentration in the particle suspension can vary greatly, and is related to the suspension concentration of the template particles. In one or more embodiments, the soluble matrix precursor concentration ranges from about 0.0005 to 0.10 wt %, or about 0.005 to about 1.0 wt %. The actual amount of precursor used will depend on the nature of the precursor, the template and the desired end product and application. FIGS. 6A-E illustrate the range of soluble matrix precursor concentration for a PMMA/silica precursor solution and demonstrate the effect of increasing precursor solution concentration according to one or more embodiments. FIG. 6A shows a PMMA colloidal crystal film in top and side views with no added silica matrix having extensive cracking and small crystalline domains. FIGS. 6B-6E show a series of SiO₂ inverse opal films (after template removal by calcination at 500° C.), in top and side views, with increasing amounts of added silica matrix (i.e.; increasing SiO₂/PMMA template ratio). The values represent mL of TEOS solution (TEOS/HCl/H₂O/EtOH) added to 20 mL of 0.125% PMMA suspension. If the concentration of matrix precursor is too low, a continuous network of matrix may not be formed (FIGS. 6B, 6C). For the current system, the TEOS level shown in FIG. 6D provided conditions for large domain, crack-free, overlayer-free inverse opal films. Increasing the silica matrix concentration further causes the formation of a continuous overlayer (FIG. 6E).

The particle suspension can consist of size-monodispersed templating particles, for ordered, periodic structures, or can consist of templating particles having a distribution of sizes, for disordered structures (without long-range order). If there is a large variation of template particle size used, then a hierarchy of pore sizes can be produced. The size of the particles for the template can range from 50 nm to 1000 nm or more. In one or more embodiments, the particle size of the templating particles can range between about 200 nm and 1000 nm. In one or more embodiments, the template particle can be up to about 2 μm, up to about 10 μm, or up to about 300 μm or even as high as about 500 μm. Porous structures having particles of up to 300 μm may be particularly suitable for applications in tissue engineering, where pore sizes of about 100 to 300 μm are well-suited for cell growth and blood vessel formation.

The templating particles can be made of various materials, so long as they are capable of assembly from solution and can be removed after assembly, if desired. By way of example, the templating particles can be colloidal polymers, such as various known latexes. Such templating particles can be removed, if desired, by thermal decomposition (burning or gasification), plasma etching or dissolution in a suitable solvent. In other embodiments, the templating particles can be metal oxides, such as colloidal silica and colloidal alumina and other metal oxides. Such templating particles can be removed, if desired, by solvent etching and dissolution.

If the colloidal template particles are not removed, a composite material of the polymer template and matrix can be used for applications such as optically-iridescent paint coatings, or mechanically-robust composite layers.

In one or more embodiments, the matrix precursor can also be capable of supramolecular self-assembly in addition to the self-assembly of the template composition. As an example, surfactant or block copolymer self-assembly can occur within the matrix material to produce a ‘mesoporous’ network, with porosity at a smaller scale than the template porosity. Therefore, pores at two distinct length scales are produced.

In one or more embodiments, the co-assembly process may be used in two or more steps to co-assemble elements of increasing size to provide a composite or related porous structure having hierarchical arrangement of particles with varying dimensions. By way of example, a co-assembly can be carried out using a particle suspension of particles on the order of 100 nm-300 m, and large polymer beads on the order of 100-500 μm, with a sol-gel matrix precursor.

The co-assembly EISA process using a simplified one-step process provides a co-assembly of templating particles and matrix, e.g., metal oxide that is crack-free and with uniform density. The co-assembly process typically does not form an overlayer, which means that the extremely high porosity of the films is also very accessible from the top surface (instead of being limited to just the sides). This is very important for applications such as catalysis, gas adsorption, fuel cells or tissue engineering. If the templating particles have a monodispersed size distribution, then highly-ordered nanoporous films will be formed, which is particularly suitable for photonic applications.

In one or more embodiments, a highly-ordered nanocomposite is obtained having significantly reduced defects, as compared to products obtained from a conventional EISA composite. There is typically a great reduction in the number and size of cracks that are formed. Macroscopic substrates (i.e., 1-10 cm size) can be coated with films that have virtually no cracks at all.

The co-assembly with the matrix material has a significant effect on the structural order of the templating particles. The colloidal crystal deposited using traditional evaporative deposition (from a solution containing no matrix material), shows significant cracking with a characteristic branched pattern at two length scales: (1) large, interconnected {111} cracks with a typical inter-crack distance of ˜10 μm, and (2) micro-cracks with a typical inter-crack distance of ˜1-2 μm (FIG. 3A). A variety of defects and micron-sized misaligned domains in these films are evident. The infiltration step further reduces the quality of the films due to the formation of an overlayer, partial filling of the cracks developed during the assembly of the template PMMA crystal, and an additional ‘glassy’ crack pattern originated from the overlayer and non-uniform infiltration (FIG. 3B). When templating particles are combined with the sol-gel matrix and allowed to co-assemble according to the one or more embodiments of the current invention, ordered domains appear to reach the size of the substrates themselves (i.e. 1-10 cm)—a factor of ×10,000-100,000 improvement over the conventional technique. When a thick layer of inverse opal is intentionally stressed and caused to crack, the resultant cracks occur at regular arrangement of 60 degrees. The regular and ordered arrangement of cracks at 60° is evidence of long-range crystalline order in the structure, implying that the film is a single crystal composed of one uniformly-oriented domain. As a result, this method can be used to produce highly-ordered nanoporous metal oxide thin films.

While not being bound by any particular mode of operation, it is theorized that the observed improvements in density uniformity and the absence of the overlayer formation is due to the presence of the soluble matrix precursor within the interstitial spaces of the colloidal crystal during assembly, so that formation of the matrix material and the inverse opal structure does not require infiltration from an external location. In addition to eliminating the infiltration step, the co-assembly EISA improves the uniformity of the colloidal crystal itself, due to the fact that the presence of a precursor modifies the wetting properties at the liquid-colloid interface, thus causing the reduction of the localized negative pressure developed in the drying suspension. In addition, the matrix material acts as a glue between the templating particles to increase the tensile strength. With this decreased capillarity and increased strength, the cracking (otherwise significant in a standard EISA film) is prevented over large length scales. An overlayer does not form because the soluble matrix material is never deposited above the layer of the colloids themselves in the co-assembly process.

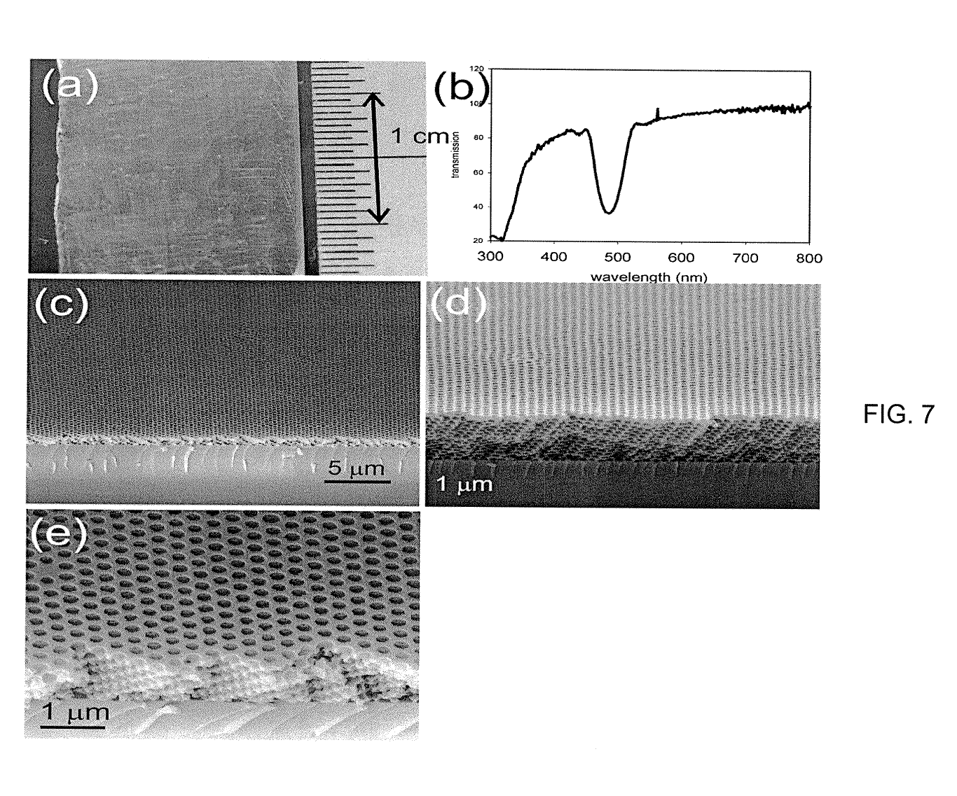

FIG. 7 shows examples of SiO₂ inverse opal films produced by the co-assembly method, using a template of 250 nm diameter polymer (PMMA) templating particles and heat-treatment at 500° C. in air to burn away the polymer template. In this case 0.15 mL of a solution of 1:1:1.5 by weight of TEOS:HCl (0.10 M):ethanol, respectively, was added to a 20 mL of the 1 wt % PMMA suspension. A 1 cm×4 cm glass slide was held vertically in the suspension and the film was deposited by drying in an oven at 60° C. on a vibration-free table, over a period of 2 d. FIG. 7A is a low magnification, optical photograph of a glass slide substrate coated in the porous film, showing the distinct color produced by the optical interference of the periodic structure. FIG. 7B shows optical absorption spectra, which indicate a peak corresponding to the Bragg diffraction condition. The absorption spectra show a peak pattern that is consistent with a single packing symmetry. The narrow width of the band is evidence of order within the crystal. FIGS. 7C-E show SEM images of porous SiO₂ inverse opal films, indicating the very high degree of order, without localized cracking, and without the formation of an overlayer (compare to FIGS. 3A-B, for a similar film produced using the conventional method).

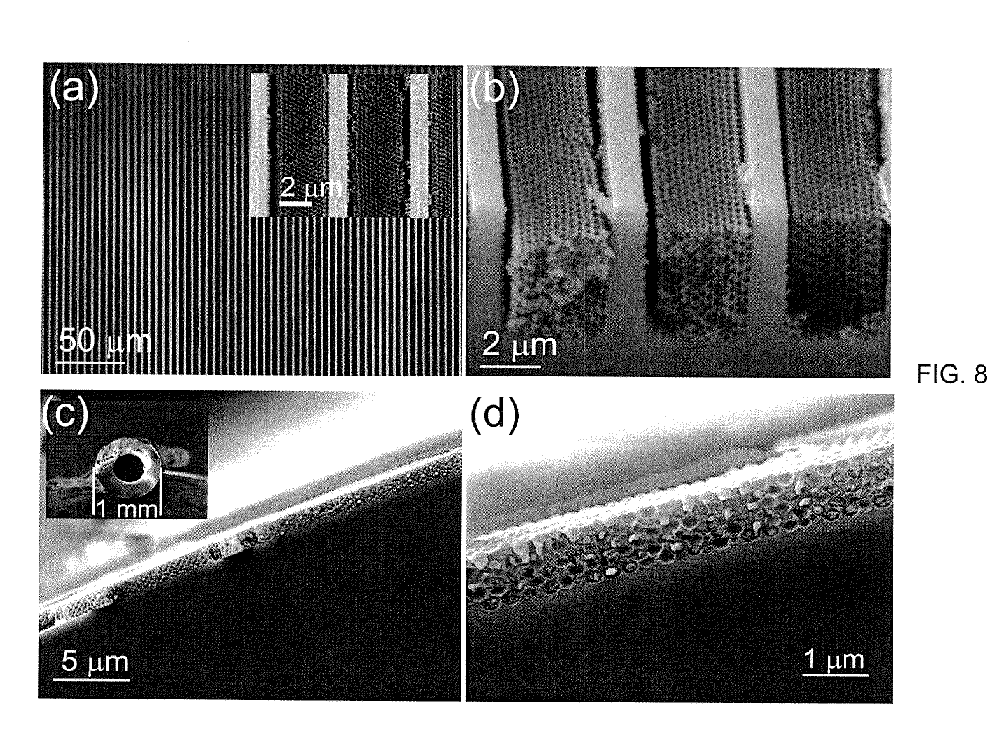

In one or more embodiments, a composite layer or an inverse opal film can be prepared on complex surfaces, such as curves, or channels. Because the resultant porous structure does not form an overlayer, it can be used to form porous structure over complex structures. FIG. 8 shows an example of a SiO₂ inverse opal film deposited within the patterned channels of a Si wafer from a top view (FIG. 8A) and a cross-sectional view (FIG. 8B). A silicon wafer was etched to provide 4 μm wide×4-5 μm deep channels. Using a TEOS precursor added to a 1 vol % suspension of 250 nm PMMA templating particles, a film was deposited by EISA at 60° C. in air with the channels oriented vertically to the deposition surface to produce (after heat-treated at 500° C. to remove the PMMA template) an inverse opal structure within the channels. Such a structure would be difficult or even impossible to prepare using conventional methods because the only surface exposed after soluble matrix infusion is coated with an overlayer. FIGS. 8C and 8E show a SiO₂/PMMA composite film (before template removal) deposited around a 1 mm diameter SiO₂ glass capillary tube (inset in FIG. 8C), to demonstrate that deposition can be made around curved surfaces.

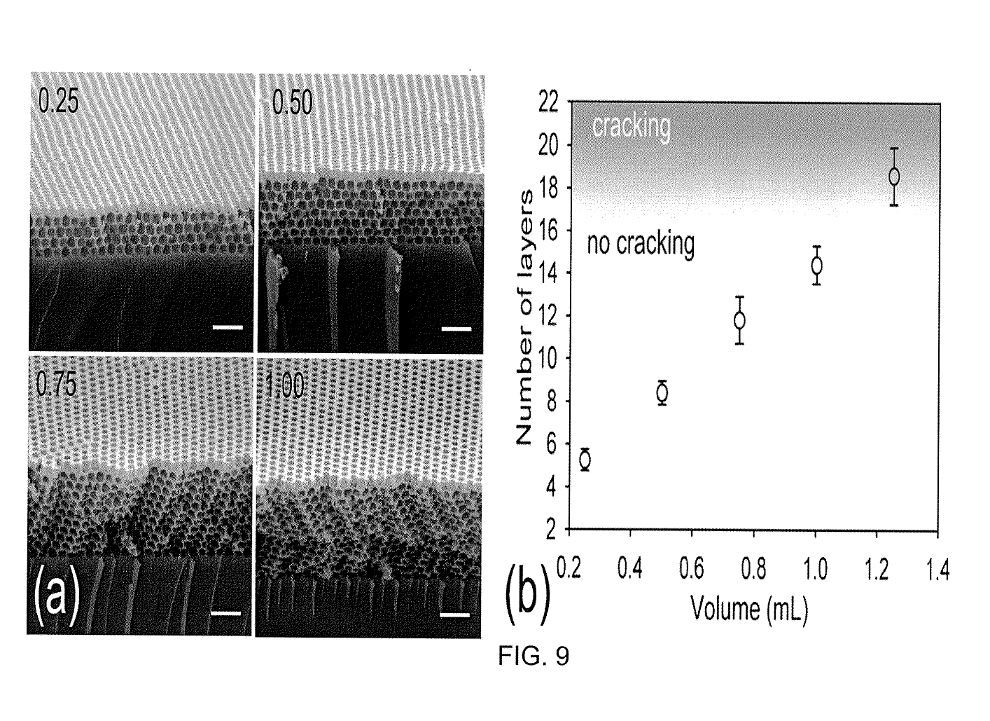

In one or more embodiments, the templating particle content (% vol. solids) of the suspension can vary over a range of about 0.10 to 3.0 vol %. The amount of particles in suspension will affect the thickness of the deposited layer, with higher concentrations of particles providing deposited films of greater thickness. In one or more embodiments, the typical colloid content is around 1-2 vol % solids content. The thickness of the inverse opal films can be controlled very precisely by adjusting the template concentration, using a fixed template/matrix ratio. FIG. 9A shows SiO₂ inverse opal films deposited at different templating particle concentrations onto a surface (values represent mL of 0.125 vol % PMMA/TEOS suspension added per 20 mL H₂O, with a fixed PMMA/TEOS weight ratio of 0.625 for each film) and FIG. 9B is a plot of thickness vs. templating particle concentration for the films of FIG. 9A. The number of layers of deposited particles increases linearly with templating particle concentration. FIG. 9B shows that no cracks forms with up to ˜18-20 sphere layers (i.e., for thicknesses up to ˜5 μm). For comparison, thin films typically have an upper threshold thickness, beyond which ‘channel’ type cracking occurs. Sol-gel SiO₂ films tend to fracture at a threshold thickness of ˜0.5 μm (10 times smaller than the co-assembled films), and colloidal crystals of similar thickness invariably crack as shown in FIG. 3A. Co-assembled films with more than 20 layers begin to fracture, with a characteristic triangular fracture extending over the entire sample (1-10 cm). Importantly, even these thick cracked films show highly increased distance between the cracks (in the order of ˜100 μm with no microcracks), thus producing defect-free regions that are 100 times larger than those in the conventional films (FIGS. 3A-B).

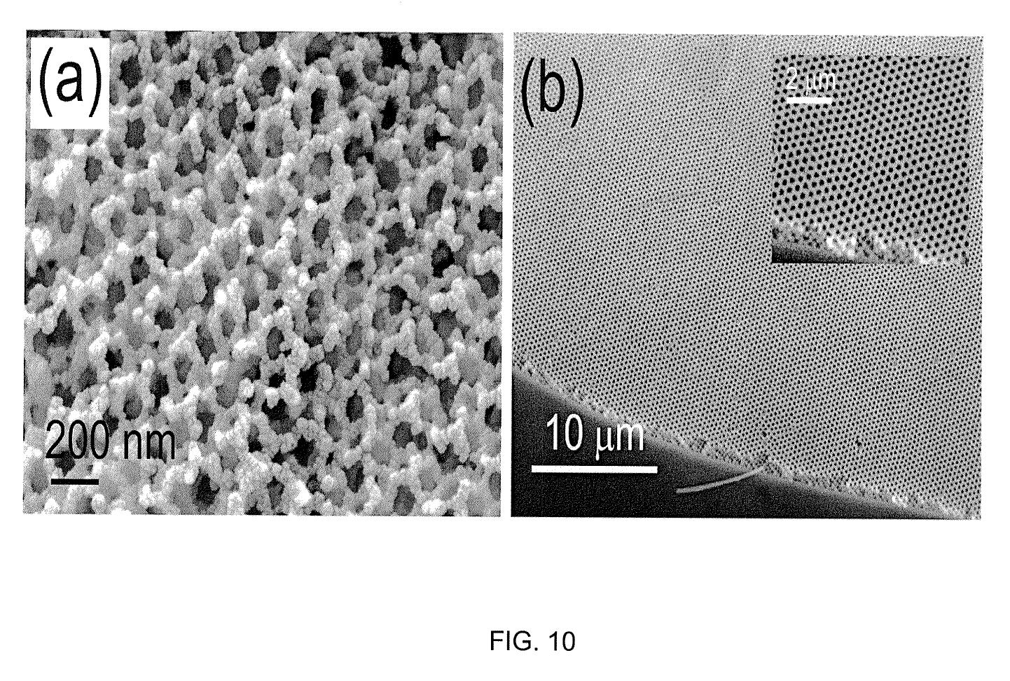

In addition, a variety of sol-gel oxide matrix precursors could be used. FIG. 10A shows a TiO₂ inverse opal films using 300 nm PMMA colloids in a solution of dihydroxybis-(ammonium lactato)titanium (IV) (TiBALDH, C₆H₁₀O₈Ti.2H₄N), after calcination. FIG. 10B shows an example of an organosilica (SiOC₂H₄) inverse opal deposited in a way similar to TEOS using a silsesquioxane alkoxide precursor ((EtO)₃Si—C₂H₄—Si(OEt)₃), as the soluble matrix materials. For those skilled in the art it is clear that the method is not limited to these exemplary materials and a wide range of metal alkoxides and polymeric precursors can be used similarly to produce ordered porous films of titania, zirconia, silica, alumina, a variety of mixed oxides, sulfides, selenides, nitrides and porous polymer scaffolds.

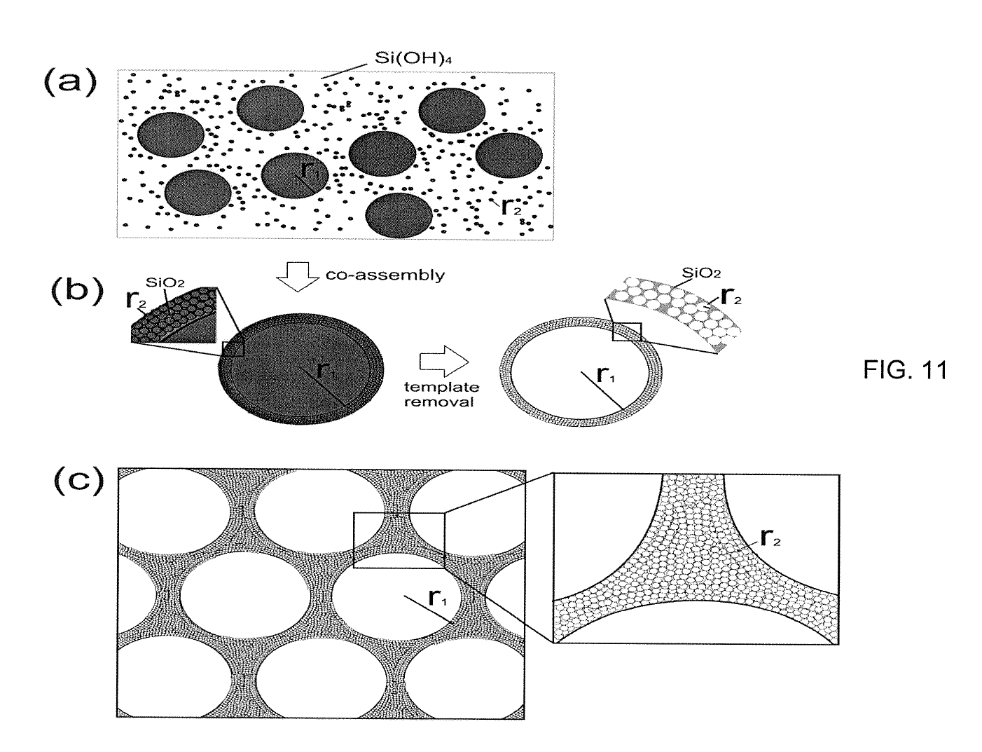

A further embodiment is the use of multiple sizes of template particles to achieve a hierarchy of pore sizes. FIG. 11 is a schematic illustration of a co-assembly process involving a soluble matrix (i.e.; Si(OH)₄) and template spheres of two different sizes. Smaller templating spheres (radius r₂) pack around a larger spheres (radius r₁). FIG. 11A shows the matrix (Si(OH)₄) and template spheres in suspension. Smaller particles are deposited onto the surface of the outer particles according to one or more methods described herein. FIG. 11B shows the co-assembled composite structure as an individual sphere shell structure, before and after template removal, showing a porous SiO₂ shell with pores of sizes r₁ and r₂. FIG. 11C shows a co-assembled inverse opal structure of many larger spheres (radius r₁) on a surface, consisting of walls having smaller pores with radius r₂.

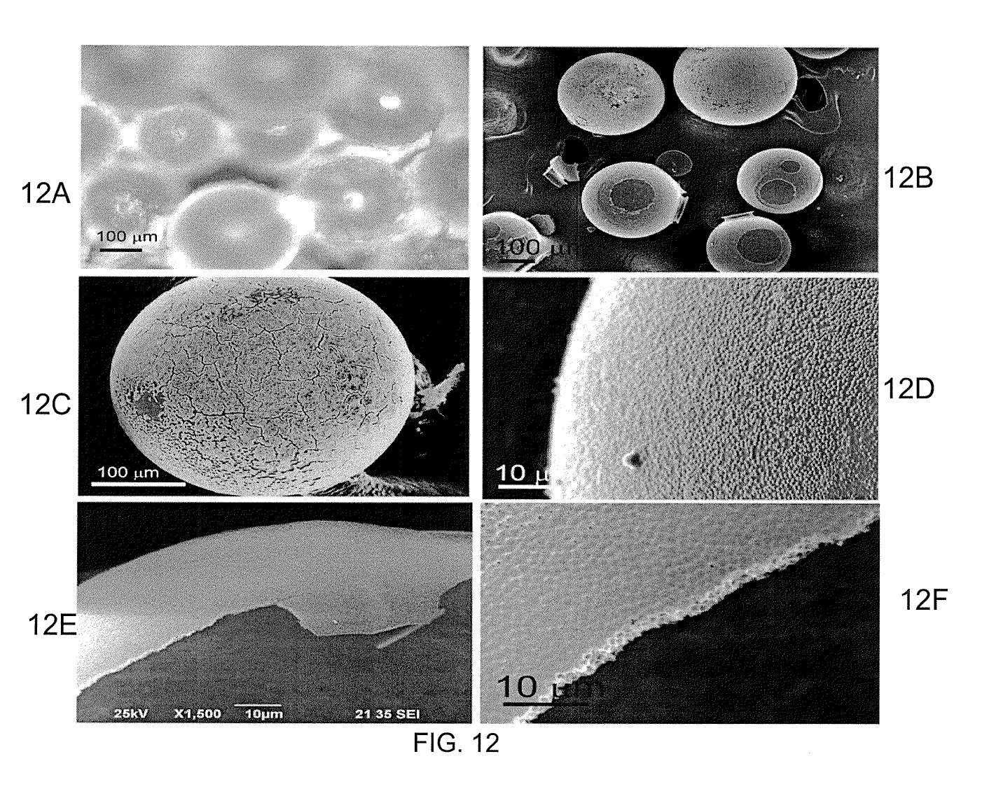

FIGS. 12A-12F are photomicrographs of 300 μm diameter porous SiO₂ shells according to the process of FIG. 11, consisting of walls having 300 nm pores. The composites are co-assembled hierarchical structures from a co-assembly of 300 nm templating PMMA spheres with large 300 micron PS spheres with a sol-gel silicate solution (TEOS solution). FIGS. 12A-D show the as-synthesized polymer template/SiO₂ composite structures, and FIGS. 12E and F show those same structures after calcination template removal, to create hierarchical porous SiO₂ shell structures. FIG. 12a is an optical image of the co-assembled structure. FIGS. 12B-D are SEM images of the co-assembled composite structures. FIGS. 12E and F are SEM images of the calcined structure, showing a fractured cross-section of the porous ‘egg shell’ SiO₂.

Porous films prepared as described herein can be further converted into a variety of materials by oxidation or reduction reactions. An example is the chemical reduction of SiO₂ at temperatures of 600-850° C. with Mg vapor to produce a composite of MgO and Si, following which the MgO can be chemically dissolved to leave behind Si in the same structure as the original SiO₂.

The process described herein provides the first synthesis of crack-free, highly-ordered inverse opal films over centimeter length scales by a simple two-step, solution-based templating particles/matrix co-assembly process. Major advantages of this co-assembly process include: (1) a great reduction in the defect population (particularly in the crack density), (2) the growth of large, highly-ordered domains via a scalable process, (3) prevention of overlayer formation and non-uniform infiltration, and (4) minimizing the number of steps involved in fabrication (i.e., avoidance of a post-assembly infiltration step provides a time/cost/quality advantage). Furthermore, these co-assembled inverse opal films are sufficiently robust and homogeneous as to allow for direct conversion, via use of morphology-preserving gas/solid displacement reactions, into inverse opal films comprised of other materials.

The ability to control pore size, pore size distribution, order and porous accessibility of nanoporous films is useful in a variety of applications. In particular, there is an important advantage in being able to combine the functionality of metal oxides with the highly porous structures, at the 10¹-10³ nm length scale that are associated with inverse opals. There are a number of important applications for these kinds of nanoporous thin films. Heterogeneous catalysts require a high surface area, and porous accessibility, for materials such as TiO₂, or as a support for catalytic surface groups or particles (such as Pt). Due to the absence of the overlayer, the high porosity of the co-assembled films is readily accessible from the top surface and makes them superior catalysts supports. For example, titania as such or as a mixed oxide catalyst can be used as catalyst for desulfurization, dehydration, dehydrogenation, esterification and transesterification reactions. It can be used as a photocatalyst for oxidation of organics and as a photosensitizer in photovoltaic cells. Along with other related compounds in sulfated form it can be used as a solid acid catalyst for alkylations, acylations, isomerizations, esterifications, nitrations, or hydrolysis.

Gas sensors and biological sensors also benefit from a high surface area, and porous accessibility, for rapid diffusion into the structure, and high sensitivity.

Drug delivery applications are another potential application for nanoporous structures in which a pharmaceutical agent is released from a nanoporous (and potentially biodegradable) scaffold at a controlled rate.

Highly-ordered nanoporous films are useful for photonic applications due to the color associated with the Bragg interference of light through the periodic variation of refractive index.

Bone tissue engineering is another application of highly porous films such as TiO₂, ZrO₂ or Al₂O₃ which have pores in the range of 100-300 μm diameter, to enable cell and blood vessel growth. The interface between a metal (i.e.; Ti) and ceramic (TiO₂), implant next to bone requires that there is a mechanical bond produced by the growth of human cells onto the implant material. This bond is particularly enhanced if a porous structure is presented to the osteoblast (bone growth) cells, to produce mineralized tissue, on the surface of the implant and to improve vascularization. Therefore, a uniform porous TiO₂ layer could be engineered to be an ideal surface structure for a biomedical implant.

Upon review of the description and embodiments of the present invention, those skilled in the art will understand that modifications and equivalent substitutions may be performed in carrying out the invention without departing from the essence of the invention. Thus, the invention is not meant to be limiting by the embodiments described explicitly above, and is limited only by the claims which follow.

The following references are hereby incorporated in their entirety by reference.
[1] Lytle, J. C.; Stein, A., Recent Progress in Synthesis and Applications of Inverse Opals and Related Macroporous Materials Prepared by Colloidal Crystal Templating. Ann. Rev. Nano Res. 2006, 1, 1-79.
[2] Stein, A.; Schroden, R. C., Colloidal crystal templating of three-dimensional ordered macroporous solids: materials for photonics and beyond. Current Opinion in Solid State and Materials Science 2001, 5, 553-564.
[3] Colvin, V. L., From Opals to Optics: Colloidal Photonic Crystals. MRS Bulletin 2001, 26, 637-641.
[4] Xia, Y.; Gates, B.; Yin, Y.; Lu, Y., Monodispersed Colloidal Spheres: Old Materials with New Applications. Advanced Materials 2000, 12, (10), 693-713.
[5] Velev, 0. D.; Jede, T. A.; Lobo, R. F.; Lenhoff, A. M., Porous silica via colloidal crystallization. Nature 1997, 389, 447-448.
[6] Holland, B. T.; Blanford, C. F.; Stein, A., Synthesis of macroporous minerals with highly ordered three-dimensional arrays of spheroidal voids. Science 1998, 281, 538-40.
[7] Brinker, C. J.; Scherer, G. W., Sol-Gel Science: The Physics and Chemistry of Sol-Gel Processing. Academic: 1990.
[8] Dimitrov, A. S.; Nagayama, K. Langmuir 1996, 12, 1303-1311
[9] Jiang, P.; Bertone, J. F.; Hwang, K. S.; Colvin, V. L., Single-crystal colloidal multilayers of controlled thickness. Chem. Mater. 1999, 11, 2132-2140.
[10] Norris, D. J.; Arlinghaus, E. G.; Meng, L.; Heiny, R.; Scriven, L. E., Opaline photonic crystals: How does self-assembly work? Adv. Mater. 2004, 16, (16), 1393-1399.
[11] Wong, S.; Kitaev, V.; Ozin, G. A., Colloidal Crystal Films: Advances in Universality and Perfection. J. Am. Chem. Soc. 2003, 125, 15589-15598.
[12] Dziomkina, N. V.; Vancso, G. J., Colloidal crystal assembly on topologically patterned templates. Soft Matter 2005, 1, (4), 265-279.
[13] Yang, P.; Deng, T.; Zhao, D.; Feng, P.; Pine, D.; Chmelka, B. F.; Whitesides, G. M.; Stucky, G. D., Hierarchically ordered oxides. Science 1998, 282, (5397), 2244-2247.
[14] Scharrer, M.; Wu, X.; Yamilov, A.; Cao, H.; Chang, R. P. H., Fabrication of inverted opal ZnO photonic crystals by atomic layer deposition. Appl. Phys. Lett. 2005, 86, 151113-5.
[15] King, J. S.; Heineman, D.; Graungnard, E.; Summers, C. J., Atomic layer deposition in porous structures: 3D photonic crystals. Appl. Surf. Sci. 2005, 244, 511-516.
[16] Blanco, A.; Chomski, E.; Grabtchak, S.; Ibisate, M.; John, S.; Leonard, S. W.; Lopez, C.; Meseguer, F.; Miguez, H.; Mondia, J. P.; Ozin, G. A.; Toader, O.; van Driel, H. M., Large-scale synthesis of a silicon photonic crystal with a complete three-dimensional bandgap near 1.5 micrometres. Nature 2000, 405, 437-440.
[17] Vlasov, Y. A.; Bo, X.-Z.; Sturm, J. C.; Norris, D. J., On-chip natural assembly of silicon photonic bandgap crystals. Nature 2001, 414, (6861), 289-293.
[18] Shea, K. J.; Loy, D. A.; Webster, O., Arylsilsesquioxane gels and related materials. New hybrids of organic and inorganic networks. J. Am. Chem. Soc. 1992, 114, 6700-6710.
[19] Kickelbick, G., Hybrid Inorganic-Organic Mesoporous Materials. Angew. Chem. Int. Ed. 2004, 43, 2-4.

What is claimed is:

1. A method of making a composite comprising:
a. providing a particle suspension comprising templating particles and a soluble matrix precursor;
b. co-depositing the templating particles and the matrix precursor on a surface as a composite assembly comprised of templating particles with an interstitial matrix.

2. The method of claim 1, wherein the templating particles are selected from the group consisting of organic polymers, silicates and metal oxides.

3. The method of claim 1, wherein the templating particles have a diameter in a range of about from 50 nm to 1000 nm.

4. The method of claim 1, wherein the templating particles have a diameter of up to about 2 μm.

5. The method of claim 1, wherein the templating particles have a diameter in a range of about 2 μm to about 500 μm.

6. The method of claim 1, wherein the templating particles are monodispersed in size.

7. The method of claim 1, wherein the composite assembly is a periodic, close-packed, defect-free structure with long-range order.

8. The method of claim 1, wherein the method of depositing comprises evaporative self-assembly.

9. The method of claim 1, wherein the method of depositing is selected from the group consisting of sedimentation, evaporative techniques, shear flow reactions, spin-coating, and filtration.

10. The method of claim 1, wherein the soluble matrix precursor is selected from the group consisting of metal oxide precursors, calcium phosphate precursors, soluble organic polymers, biopolymers and polymer precursors.

11. The method of claim 1, wherein the concentration of templating particles and soluble matrix precursor in the particle suspension is selected to provide a substantially crack-free composite assembly that is substantially free of an overlayer of interstitial matrix material.

12. The method of claim 1, wherein the concentration of templating particles and soluble matrix precursor in the particle suspension is selected to provide a substantially crack-free composite assembly that comprises an overlayer of interstitial matrix material.

13. The method of claim 1, wherein the soluble matrix precursor content ranges from about 0.005 wt % to about 1.0 wt %.

14. The method of claim 1, wherein the templating particle content ranges from about 0.10 vol % to about 3.0 vol %.

15. The method of claim 1, wherein the templating particles comprise particles of different sizes.

16. The method of claim 15, wherein the smaller templating particles are on the range of one to two orders of magnitude smaller than the larger templating particles.

17. The method of claim 1, further comprising:
removing the templating particles to provide an inverse porous structure.

18. A composite comprising:
a colloidal crystalline structure composed of periodic, close-packed templating particles and an interstitial matrix, wherein the crystalline structure comprises ordered domains greater than 100 μm.

19. The composite of claim 18, wherein the crystalline structure comprises ordered domains greater than 500 μm.

20. The composite of claim 18, wherein the crystalline structure comprises ordered domains in the range of about 100 μm to about 10 cm.

21. The composite of claim 18, wherein the colloidal crystalline structure is substantially crack-free.

22. The composite of claim 18, wherein the interstitial matrix is selected from the group consisting of organic polymers, calcium phosphate precursors, biopolymers and metal oxides.

23. The composite of claim 18, wherein the metal oxide precursor is single metal oxide or a mixed metal oxide selected from the group consisting of SiO₂, TiO₂, Al₂O₃, ZrO₂ and GeO₂.

24. The composite of claim 18, wherein the soluble organic polymer is selected from the group consisting of polyacrylic acids, polymethylmethacrylates, cellulose, polydimethyl siloxane, polypyrrole and agarose.

25. The composite of claim 18, wherein the colloidal crystalline structure comprises templating particles having a diameter in a range of about from 50 nm to 1000 nm.

26. The composite of claim 18, wherein the colloidal crystalline structure comprises templating particles having a diameter of up to about 2 μm.

27. The composite of claim 18, wherein the colloidal crystalline structure comprises templating particles having a diameter in the range of about 2 μm to about 500 μm.

28. The composite of claim 18, wherein the templating particles comprise particles of different sizes.

29. The composite of claim 18, wherein the smaller templating particles are on the range of one to two orders of magnitude smaller than the larger templating particles.

30. The composite of claim 18, wherein the ratio of templating particle to interstitial matrix is in the range of about 2:1 to about 1:2 on a vol/weight basis.

31. The composite of claim 18, wherein the colloidal crystalline structure is substantially free of an overlayer of interstitial matrix material.

32. An inverse opal porous layer, comprising:
an interstitial matrix defining pores, wherein the layer is substantially crack free and the pore structure comprises ordered domains greater than 100 μm.

33. The inverse opal layer of claim 32, wherein the pore structure comprises ordered domains greater than 500 μm.

34. The inverse opal layer of claim 32, wherein the pore structure comprises ordered domains in the range of about 100 μm to about 10 cm.

35. The inverse opal layer of claim 32 wherein the pores have a diameter in a range of about from 50 nm to 1000 nm.

36. The inverse opal layer of claim 32, wherein the pores have a diameter of up to about 2 μm.

37. The inverse opal layer of claim 32, wherein the pores have a diameter in the range of about 2 μm to about 500 μm.

38. The inverse opal layer of claim 32, wherein the matrix is selected from the group consisting of metal oxides, organic polymers, calcium phosphates and block copolymers.

39. The inverse opal layer of claim 32, wherein the matrix comprises nanoparticles that are less than about 10 nm in diameter.

40. The inverse opal layer of claim 32, wherein the matrix comprises nanoparticles that are less than about 5 nm in diameter.

41. The inverse opal layer of claim 32, wherein the pore structure has a hierarchy of pore sizes, with large macropores in the range 1 μm to around 2 mm.

42. A device selected from the group consisting of a photonic device, a sensor, a fuel cell, a drug release and a catalyst support comprising the inverse opal porous structure of claim 32.

43. A scaffold for tissue engineering comprising inverse opal porous structure of claim 37.
